(19) United States
(12) Patent Application Publication
(10) Pub. No.: US 2022/0348603 A1
(43) Pub. Date: Nov. 3, 2022

(54) ISOMERIZATION FEATURE-BASED METHOD FOR PURIFYING PUNICALAGIN

(71) Applicant: XINJIANG TECHNICAL INSTITUTE OF PHYSICS & CHEMISTRY, CHINESE ACADEMY OF SCIENCES, Urumqi (CN)

(72) Inventors: Haji Akber AISA, Urumqi (CN); Guangying SUN, Urumqi (CN); Yongxin ZHAO, Urumqi (CN); Munire ABUDUAINI, Urumqi (CN); Guliqire ADILI, Urumqi (CN)

(73) Assignee: XINJIANG TECHNICAL INSTITUTE OF PHYSICS & CHEMISTRY, CHINESE ACADEMY OF SCIENCES, Urumqi (CN)

(21) Appl. No.: 17/624,259

(22) PCT Filed: Mar. 20, 2020
(86) PCT No.: PCT/CN2020/082035

§ 371 Date: Dec. 31, 2021

(30) Foreign Application Priority Data
Aug. 2, 2019 (CN) 201910711349.9

(51) Int. Cl. C07H 1/08; C07H 19/01

(52) U.S. Cl. CPC C07H 1/08; C07H 19/01

(57) ABSTRACT

An isomerization feature-based method for purifying and preparing punicalagin is provided, wherein pomegranate peel extract is used as a raw material. The isomerization feature-based method avoids impurities contained in the punicalagin based on structural characteristics, for example, the punicalagin in the pomegranate peel extract has two mutually convertible isomers. In the isomerization feature-based method, a pilot-scale preparative liquid chromatography is used to obtain a large amount of the punicalagin having a purity higher than 98% from a complex pomegranate peel extract. The isomerization feature-based method is simple, an obtained punicalagin has high purities and a preparation is in a massive scale. The isomerization feature-based has a strong reference value for purification and preparation of compounds with isomerization features.

### CROSS REFERENCE TO THE RELATED APPLICATIONS

This application is the national stage entry of International Application No. PCT/CN2020/082035, filed on Mar. 30, 2020, which is based upon and claims priority to Chinese Patent Application No. 201910711349.9 filed on Aug. 2, 2019, the entire contents of which are incorporated herein by reference.

### TECHNICAL FIELD

The invention relates to an isomerization feature-based method for purifying punicalagin.

### BACKGROUND

Obtaining a large amount of high-purity monomer compounds from complex natural product medicinal materials or extracts has always been a hot spot and challenging issue. Natural products have many components and very complex structural composition. A single compound is often neighbored by compounds with highly similar structures, which causes extremely serious interference to the target compound separation. This interference is often called matrix effect. In traditional column chromatography, liquid chromatography, and countercurrent chromatography, this matrix effect often brings significant difficulties to the separation of pure compounds, or even causes failure of obtaining products with qualified purity. Therefore, it is very important to invent a separation method that can effectively avoid the matrix effect and can be applied to simple chromatographic separation methods, so as to improve the purification efficiency of monomer compounds from natural products.

Isomers, as being widely found in natural products, are compounds with same molecular weight but different structures. There are many kinds of isomers, and some of them have special structures e.g., certain epimers and cis-trans isomers, which can be transformed into each other under certain conditions. Currently, quite mature and intensive studies have been done to this isomerization reaction. However, introduction of this isomerization feature into separation and purification field to solve the matrix effect issue in purification of isomerizable compounds is a brand-new concept and has not been reported in any literature so far. Such a method can be called as an isomerization method of purification.

The basic principle of the isomerization method of purification is that an isomerizable compound usually shows two or more peaks on the chromatogram. The component containing one of the isomers and co-eluted impurities is collected from chromatography and transformed rapidly into a component comprising all isomers under certain conditions (one-dimensional separation). When the component is analyzed by liquid chromatography, peaks of all isomers are presented on the chromatogram. The component is secondly purified with the same method used in one-dimensional separation. All isomer components other than the component of the original isomer are collected selectively, which can effectively avoid the impurities embedded in the original isomer. Then, under certain conditions, the obtained components are transformed into the mixture of all isomers without any impurity.

Punicalagin is a kind of plant polyphenol with ellagic acid as its mother nucleus and rich in pomegranate peel medicinal materials, which has presented various biological activities of anti-inflammatory, antibacterial, antiviral, preventing the proliferation of cancer cell, making it a potential compound for medicine. In the structure of punicalagin, there exists a glucoside with one end carbon, which can cause two anomers, namely, α-punicalagin and β-punicalagin. The two isomers of punicalagin can mutually transformed on the chromatography. At present, the purification of punicalagin mainly rely on the methods based on column chromatography, liquid chromatography and countercurrent chromatography, as well as the combination of them.

Because the matrix effect is very complicated in natural products, the purification of punicalagin is very difficult, punicalagin in high-purity can be prepared in a low amount, or even it is impossible to prepare punicalagin with extremely high purity. Utilizing the isomerizable characteristics of punicalagin, the isomerization feature-based purification method is developed.

By using pilot-scale chromatography, large amounts of punicalagin in high purity was prepared with the feature-based purification method. The key point of the present invention is the development of isomerization feature-based purification method and its application in the batch preparation of high-purity punicalagin, which is of great significance for the research and development of new drugs based on punicalagin. The somerization feature-based purification method is suitable for purifying isomerizable compounds, which is innovative and demonstrative.

### SUMMARY

The purpose of the present invention is to provide an isomerization feature-based method for purifying punicalagin. Utilizing the isomerizable characteristics of punicalagin, impurities co-eluted with punicalagin in pomegranate peel extract can be effectively removed by isomerization feature-based method. By using pilot-scale preparative liquid chromatography, grams of punicalagin with the purity higher than 98% were prepared from the complex extract of pomegranate peel. The method is simple, effective and economic, which is valuable for the purification and preparation of compounds with isomerization characteristics.

The isomerization feature-based method for purifying punicalagin of the present invention comprises the following steps:

a) taking a reversed-phase chromatographic column, wherein the mobile phase is consisted of methanol and 0.01%-5% (v/v) formic acid water in a volume ratio of 1-5:9-5, the flow rate is controlled at 50-300 mL/min, and the UV detection wavelength is 220-280 nm and 350-380 nm, the column is equilibrated with mobile phase for 3-30 min before use;

b) weighing pomegranate peel extract, dissolving them in water, taking the supernatant after centrifuging, and loading it into the reversed-phase chromatographic column, two components around the peaks of α-punicalagin and β-punicalagin were collected individually, and then concentrated by rotary evaporator at 30-50° C.;

c) reloading the concentrated component collecting from the peak of α-punicalagin obtained in step b) into a reversed-phase chromatographic column for the separation under the same conditions as step a), and two components around chromatographic peaks of α-punicalagin and β-punicalagin were collected respectively, wherein the component collected around the peak of β-punicalagin is concentrated by rotary evaporator at a temperature of 30-50° C. and lyophilized as the powder of punicalagin with the purity higher than 98%, as detected by high-performance liquid chromatography; the component collected around of the peak of α-punicalagin is concentrated by rotary evaporator at a temperature of 30-50° C., and circularly separated under the same chromatographic condition as step a) to prepare high-purity punicalagin;

and/or,

reloading the concentrated component collecting around the peak of β-punicalagin obtained in step b) into a reversed-phase chromatographic column under the same conditions as step a), and two components around the peaks of α-punicalagin and β-punicalagin were collected, wherein the component collected around α-punicalagin is concentrated by rotary evaporator at a temperature of 30-50° C. and reloaded into the reversed-phase chromatographic column in step a), two components around the peaks of α-punicalagin and β-punicalagin are collected respectively, wherein the component collected around α-punicalagin is concentrated by rotary evaporator at a temperature of 30-50° C. and lyophilized as pale yellow powder of punicalagin with the purity higher than 98%, as detected by high-performance liquid chromatography; the component collected around the peak of β-punicalagin is concentrated by rotary evaporator at a temperature of 30° C.-50° C., and circularly purified under the same chromatographic condition as step a) to prepare high-purity punicalagin.

In the method for purifying punicalagin according to the present invention, preferably, in step b, 0.1-10 g of pomegranate peel extract is weighed and dissolved in 10-50 mL of water.

In the method for purifying punicalagin according to the present invention, preferably, in step b, the solution is centrifuged at a rotation speed of 5000-10000 revolutions per minute for 3-5 minutes.

The method for purifying punicalagin according to the present invention is characterized that the concentration is performed to reduce the volume to 10-50 mL. Further preferably, the concentration is performed using a rotary evaporator.

According to any one of the methods for purifying punicalagin of the present invention described above is characterized that, as a preferred mode, the isomerization feature-based method for purifying punicalagin of the present invention is carried out as following steps:

a) taking a pilot-scale reverse-phase chromatographic column, wherein the mobile phase is consisted of methanol and 0.1% formic acid water in a volume ratio of 12:88, the flow rate is controlled at 180 mL/min, the temperature is set at room temperature, and the UV detection wavelength is 254 nm and 366 nm, the column is equilibrated for 15 min before use;

b) weighing 6 g of pomegranate peel extract, dissolving them in 20 mL of water, taking the supernatant after centrifuging the extract solution at 10,000 rpm for 5 minutes and loading it into the equilibrated pilot-scale reversed-phase chromatographic column through a six-way valve, collecting two components around the two chromatographic peaks around α-punicalagin and β-punicalagin, then concentrating the two components to 20 mL with a rotary evaporator at 50° C. respectively;

c) reloading the concentrated component collecting around the peak of α-punicalagin obtained in step b) into a pilot-scale reversed-phase chromatographic column for the secondary separation under the same conditions as step a), and collecting two components around the chromatographic peaks around the peak of α-punicalagin and β-punicalagin respectively, wherein the component collected around the peak of β-punicalagin is concentrated t with a rotary evaporator at a temperature of 39° C. and lyophilized as 137 mg pale yellow powder of punicalagin with the purity higher than 98% after detected by high-performance liquid chromatography; concentrating the component collected around the peak of α-punicalagin to a volume of 20 mL with a rotary evaporator at a temperature of 50° C., and circularly loading the concentrated component under the same chromatographic condition as step a) to prepare high-purity punicalagin;

or,

reloading the concentrated component collecting around the peak of β-punicalagin obtained in step b) into a pilot-scale reversed-phase chromatographic column under the same conditions as step a), and collecting the two chromatographic peaks around the peaks of α-punicalagin and β-punicalagin respectively, wherein the component collected around the peak of α-punicalagin is the qualified product; concentrating the component collected around the peak of β-punicalagin to a volume of 20 mL with a rotary evaporator at a temperature of 50° C., reloading the concentrated component into the pilot-scale reversed-phase chromatographic column in step a), collecting the components around the two chromatographic peaks at the positions of α-punicalagin and β-punicalagin respectively, wherein the component collected around the peak of α-punicalagin is the qualified product; combining the two components collected around the peak of α-punicalagin, concentrating the combined components with a rotary evaporator at a temperature of 39° C., lyophilizing the concentrated components to obtain 280 mg pale yellow powder of punicalagin with a purity of higher than 98% after detected by high-performance liquid chromatography; concentrating the component collected around the peak of β-punicalagin to a volume of 20 mL with a rotary evaporator at a temperature of 50° C., and circularly loading the concentrated component under the same chromatographic condition as step a) to prepare high-purity punicalagin.

The isomerization feature-based method for purifying punicalagin of the present invention is characterized in that the chromatographic column used in the method is a reversed-phase C18 chromatographic column, wherein a dosing ring with a volume of 30 mL is used for loading samples, which is larger than the sample volume i.e. 20 mL.

The isomerization feature-based method for purifying punicalagin of the present invention is an isomerization purification method developed based on the isomerizable characteristics of punicalagin, and it is used for batch preparation of high-purity punicalagin. The method is simple and practical, can be used for massive-scale preparation. The purity of the obtained punicalagin is higher than 98%. The established isomerization feature-based method is suitable for purifying isomerizable compounds, which is innovative and demonstrative.

### BRIEF DESCRIPTION OF THE DRAWINGS

FIG. 1 is a pilot-scale reversed-phase chromatogram of the pomegranate peel extract involved in the present invention;

FIG. 2 is a preparative chromatogram of the α-punicalagin component 1 involved in the present invention after being concentrated and reloaded onto the pilot-scale reversed-phase chromatographic column;

FIG. 3 is a liquid chromatographic identification diagram of the β-punicalagin component 3 obtained in the present invention;

FIG. 4 is a preparative chromatogram of the β-punicalagin component 4 involved in the present invention after being concentrated and reloaded onto the pilot-scale reversed-phase chromatographic column;

FIG. 5 is a liquid chromatographic identification diagram of the combined α-punicalagin component 5 and α-punicalagin component 7 obtained in the present invention.

### DETAILED DESCRIPTION OF THE EMBODIMENTS

The present invention will be further described in detail below in conjunction with specific embodiments.

### Example 1

a. A pilot-scale reversed-phase chromatographic column was taken, wherein the column had a size of 250 mm (column length)×80 mm (inner diameter); its filler had a particle size of 10 μm; the mobile phase was consisted of methanol and 0.1% formic acid water in a volume ratio of 12:88; the flow rate was kept at 180 mL/min; the temperature was set at room temperature, and the UV detection wavelength was 254 nm and 366 nm, the column was equilibrated for 15 min, and ready for use.

b. 6 g of pomegranate peel extract was weighed, wherein the content of punicalagin was about 32%. The extract was dissolved in 20 mL of water and centrifuged at 10,000 rpm for 5 minutes. The supernatant was collected and loaded into the equilibrated pilot-scale reversed-phase chromatographic column through a six-way valve, as shown in FIG. 1. The component named as component 1 in the time range from 10 min to 15 min i.e. α-punicalagin was collected in a component tank and concentrated to a volume of 20 mL with a rotary evaporator at 50° C. respectively.

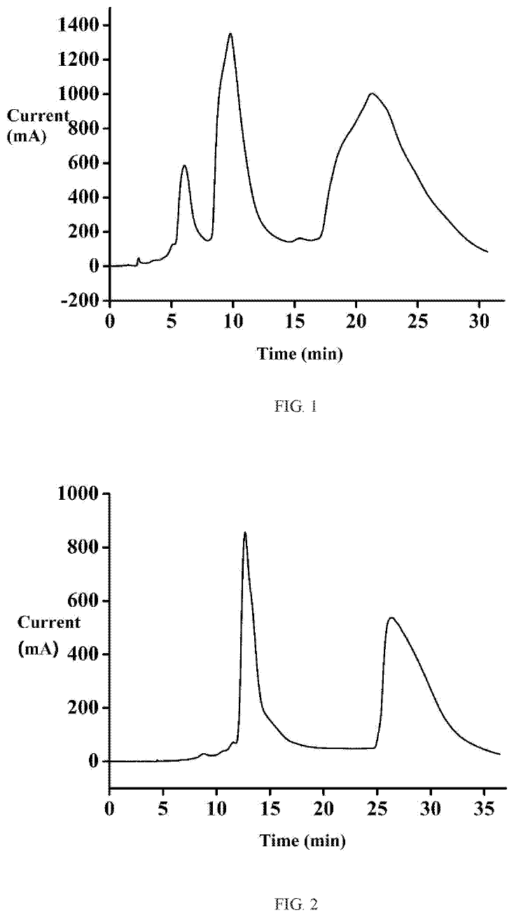

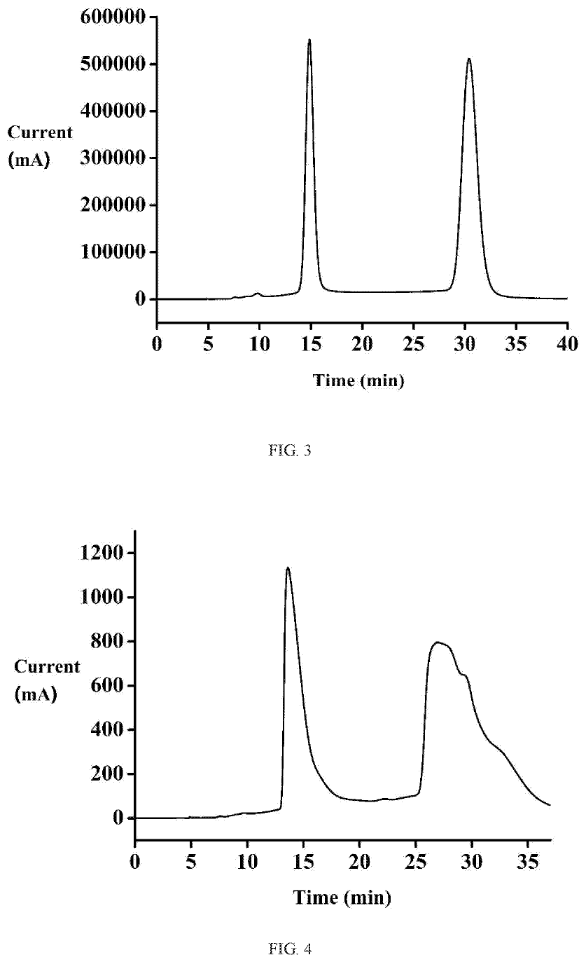

c. The component 1 was reloaded into the balanced pilot-scale reversed-phase chromatographic column according to step a. Its chromatogram is shown in FIG. 2. The components named as component 2 in the time range from 12.5 min to 20 min (around α-punicalagin) and component 3 in the time range from 25 to 35 minutes (around s-punicalagin) were collected in different component tanks respectively. The component 3 was concentrated with a rotary evaporator at a temperature of 39° C., lyophilized to obtain pale yellow powder. Such powder was weighed 137 mg in total and showed a purity of higher than 98% after detected by high-performance liquid chromatography, as shown in FIG. 3. The component 2 was concentrated to a volume of 20 mL with a rotary evaporator at a temperature of 50° C., and circularly loaded under the same chromatographic condition as step a to prepare high-purity punicalagin.

### Example 2

a. A pilot-scale reversed-phase chromatographic column was taken, wherein the column had a size of 250 mm (column length)×80 mm (inner diameter); its filler had a particle size of 10 μm; the mobile phase was consisted of methanol and 0.1% formic acid water in a volume ratio of 12:88; the flow rate was kept at 180 mL/min; the temperature was set at room temperature, and the UV detection wavelength was 254 nm and 366 nm, the column was equilibrated for 15 min, and ready for use.

b. 6 g of pomegranate peel extract was weighed, wherein the content of punicalagin was about 32%. The extract was dissolved in 20 mL of water and centrifuged at 10,000 rpm for 5 minutes.

The supernatant was collected and loaded into the balanced pilot-scale reversed-phase chromatographic column through a six-way valve, as shown in FIG. 1. The component named as component 4 around β-punicalagin (ranging from 17 min to 30 min) was collected in a component tank and concentrated to a volume of 20 mL with a rotary evaporator at 50° C. respectively.

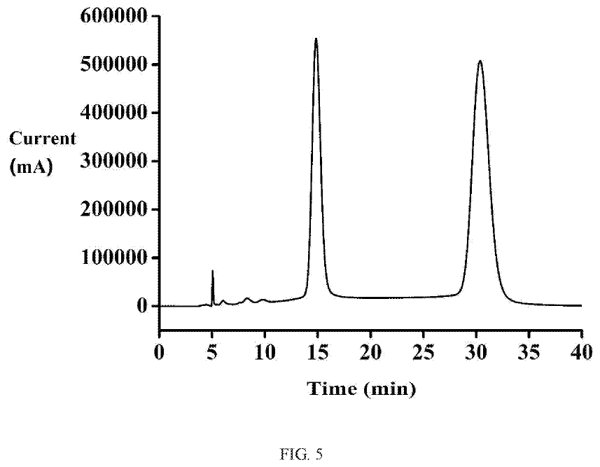

c. The component 4 was reloaded into the balanced pilot-scale reversed-phase chromatographic column according to step a. Its chromatogram is shown in FIG. 4. The components named as component 5 in the time range from 12.5 min to 20 min (around α-punicalagin) and component 6 in the time range from 25 to 35 minutes (around β-punicalagin) were collected in different component tanks respectively. The n component 5 was the qualified product.

The component 6 was concentrated to a volume of 20 mL with a rotary evaporator at a temperature of 50° C., and reloaded into the balanced pilot-scale reversed-phase chromatographic column according to step a. Its chromatogram is also shown in FIG. 4. The components named as component 7 in the time range from 12.5 min to 20 min (around α-punicalagin) and component 8 in the time range from 25 to 35 minutes (around β-punicalagin) were collected in different component tanks respectively. The α- component 7 was the qualified product. It was combined with the component 5, and concentrated with a rotary evaporator at a temperature of 39° C., then lyophilized to obtain pale yellow powder. Such powder was weighed 280 mg in total and showed a purity of higher than 98% after detected by high-performance liquid chromatography, as shown in FIG. 5. The component 8 was concentrated to a volume of 20 mL with a rotary evaporator at a temperature of 50° C., and circularly loaded under the same chromatographic condition as step a to prepare high-purity punicalagin.

Besides methanol-0.1% formic acid water mixture, the mobile phase of the reversed-phase chromatographic column used in the Examples can also be acetonitrile-0.1% formic acid water mixture, tetrahydrofuran-0.1% formic acid water mixture, methanol-acetonitrile-0.1% formic acid water mixture, methanol-tetrahydrofuran-0.1% formic acid water mixture, acetonitrile-tetrahydrofuran-0.1% formic acid water mixture, methanol-acetonitrile-tetrahydrofuran-0.1% formic acid water mixture. The content of 0.1% formic acid water should be controlled between 50% and 95% in the mobile phase.

In addition to the reversed-phase C18 chromatographic column, other reversed-phase chromatographic columns including reversed-phase C8 column, reversed-phase C4 column, reversed-phase C30 column, reversed-phase C2 column, reversed-phase cyano column, reversed-phase polystyrene column can also be used as the reversed-phase chromatographic columns of the Examples.

Besides the column length of 250 mm, the inner diameter of 80 mm, and the filler particle size of 10 μm, the reversed-phase chromatographic column involved in the Examples, can also has a column length of 50 mm-1000 mm, an inner diameter of 2.1 mm-2000 mm, and a filler particle size of 1.2 μm-500 μm.

What is claimed is:

1. An isomerization feature-based method for purifying punicalagin, comprising the following steps of:
a) taking a reversed-phase chromatographic column, wherein a mobile phase is consisted of methanol and 0.01%-5% formic acid water in a volume ratio of 1-5:9-5, a flow rate is controlled at 50-300 mL/min, and a UV detection wavelength is 220-280 nm and 350-380 nm, and the reversed-phase chromatographic column is balanced for 3-30 min to obtain a balanced reversed-phase chromatographic column;
b) weighing a pomegranate peel extract, dissolving the pomegranate peel extract in water, taking a supernatant after centrifuging an extract solution and loading the supernatant into the balanced reversed-phase chromatographic column, collecting two components around two chromatographic peaks around α-punicalagin and β-punicalagin respectively, then concentrating the two components at 30° C.-50° C. respectively to obtain two concentrated components;
c) reloading the two concentrated components at a position of the α-punicalagin obtained in step b) into the reversed-phase chromatographic column under identical conditions as step a), and collecting the two components of the two chromatographic peaks around the α-punicalagin and the β-punicalagin respectively, wherein a first component of the two components collected around a chromatographic peak of the β-punicalagin is concentrated by a rotary evaporator at a temperature of 30° C.-50° C. and lyophilized to obtain g powder of the punicalagin with a purity of higher than 98% after detected by a high-performance liquid chromatography; concentrating a second component of the two components collected from a chromatographic peak of the α-punicalagin by the rotary evaporator at a temperature of 30° C.-50° C., and circularly loading the two concentrated components under an identical chromatographic condition as step a) to prepare high-purity punicalagin;
and/or,
reloading the two concentrated components collected from the chromatographic peak of the β-punicalagin obtained in step b) into the reversed-phase chromatographic column under identical conditions as step a), and collecting the two components from the two chromatographic peaks around the α-punicalagin and the β-punicalagin respectively, wherein the second component of the two components collected from the chromatographic peak of the α-punicalagin is a qualified product; concentrating the first component of the two components collected from the chromatographic peak of the β-punicalagin by the rotary evaporator at the temperature of 30° C.-50° C., reloading the two concentrated components into the reversed-phase chromatographic column of step a), collecting the two components from the two chromatographic peaks of the α-punicalagin and the β-punicalagin respectively, wherein the second component of the two components collected from the chromatographic peak of the α-punicalagin is the qualified product; combining the second component collected from the chromatographic peak of the α-punicalagin and the first component collected from the chromatographic peak of the β-punicalagin in step c) to obtain combined components, concentrating the combined components by the rotary evaporator at the temperature of 30° C.-50° C. to obtain concentrated combined components, lyophilizing the concentrated combined components to obtain a pale yellow powder of the punicalagin with a purity of higher than 98% after detected by the high-performance liquid chromatography; concentrating the first component collected from the chromatographic peak of the β-punicalagin at the temperature of 30° C.-50° C., and circularly loading the two concentrated components under the identical chromatographic condition as step a) to prepare the high-purity punicalagin.

2. The isomerization feature-based method according to claim 1, wherein in step b), 0.1-10 g of the pomegranate peel extract is weighed and dissolved in 10-50 mL of water.

3. The isomerization feature-based method according to claim 1, wherein in step b), the extract solution is centrifuged at a rotation speed of 5000-10000 revolutions per minute for 3-10 minutes.

4. The isomerization feature-based method according to claim 1, wherein a concentration is performed to reduce a volume to 10-50 mL.

5. The isomerization feature-based method according to claim 1, wherein a concentration is performed using the rotary evaporator.

6. A method for purifying punicalagin according to claim 1, comprising following steps of:
a) taking a pilot-scale reverse-phase chromatographic column, wherein the mobile phase is consisted of the methanol and 0.1% formic acid water in a volume ratio of 12:88, the flow rate is controlled at 180 mL/min, the temperature is set at room temperature, and the UV detection wavelength is 254 nm and 366 nm, the pilot-scale reverse-phase chromatographic column is balanced for 15 min and ready for use to obtain a balanced pilot-scale reverse-phase chromatographic column;
b) weighing 6 g of the pomegranate peel extract, dissolving the 6 g of the pomegranate peel extract in 20 mL of water, taking the supernatant after centrifuging the extract solution at 10,000 rpm for 5 minutes and loading the supernatant into the balanced pilot-scale reversed-phase chromatographic column through a six-way valve, collecting the two components from the two chromatographic peaks of the α-punicalagin and the β-punicalagin, then concentrating the two components to a volume of 20 mL with a rotary evaporator at 50° C. respectively to obtain two concentrated components;
c) reloading the second component of the two concentrated components collected from the chromatographic peak of the α-punicalagin obtained in step b) into the pilot-scale reversed-phase chromatographic column under the identical conditions as step a), and collecting the two components from the two chromatographic peaks around the α-punicalagin and the β-punicalagin respectively, wherein the first component collected from the chromatographic peak of the β-punicalagin is the qualified product; concentrating the qualified product with the rotary evaporator at a temperature of 39° C. to obtain a concentrated product and lyophilizing the concentrated product to obtain 137 mg of the pale yellow powder of the punicalagin with a purity higher than 98% after detected by the high-performance liquid chromatography; concentrating the second component collected from the chromatographic peak of α-punicalagin to a volume of 20 mL with the rotary evaporator at a temperature of 50° C., and circularly loading the two concentrated components under the identical chromatographic condition as step a) to prepare the high-purity punicalagin;
or,
reloading the two concentrated components collected from the chromatographic peak of β-punicalagin obtained in step b) into the pilot-scale reversed-phase chromatographic column under the identical conditions as step a), and collecting the two components from the two chromatographic peaks of the α-punicalagin and the β-punicalagin respectively, wherein the second component collected from the chromatographic peak of the α-punicalagin is the qualified product; concentrating the second component collected by cutting at a position of the β-punicalagin to the volume of 20 mL with the rotary evaporator at the temperature of 50° C., reloading the two concentrated components into the pilot-scale reversed-phase chromatographic column of step a), collecting the two components from the two chromatographic peaks of the α-punicalagin and the β-punicalagin respectively, wherein the second component collected from the chromatographic peak of the α-punicalagin is the qualified product; combining the second component collected from the chromatographic peak of the α-punicalagin, and the first component collected from the chromatographic peak of the β-punicalagin in step c) to obtain the combined components, concentrating the combined components at the temperature of 39° C., lyophilizing the two concentrated components to obtain 280 mg of the pale yellow powder of the punicalagin with a purity of higher than 98% after detected by the high-performance liquid chromatography; concentrating the first component collected from the chromatographic peak of the β-punicalagin to the volume of 20 mL with the rotary evaporator at the temperature of 50° C., and circularly loading the two concentrated components under the identical chromatographic condition as step a) to prepare the high-purity punicalagin.

7. The isomerization feature-based method according to claim 4, wherein the concentration is performed using the rotary evaporator.

8. The method according to claim 6, wherein in step b), 0.1-10 g of the pomegranate peel extract is weighed and dissolved in 10-50 mL of water.

9. The method according to claim 6, wherein in step b), the extract solution is centrifuged at a rotation speed of 5000-10000 revolutions per minute for 3-10 minutes.

10. The method according to claim 6, wherein a concentration is performed to reduce the volume to 10-50 mL.

11. The method according to claim 6, wherein a concentration is performed using the rotary evaporator.
